SEQUENCE LISTING (5 sequences)

SEQ ID NO: 1 - DNA, 660 bp, Kagoshima Berkshire
gagaccagcaaatactatgtgaccatcattgatgccccaggacacagagacttcatcaaaaacatgattacaggcacatcccaggctgactgtgctgtcctgattgttgctgctggtgttggtgaatttgaagctggtatctccaagaacgggcagacccgcgagcatgctcttctggcttacaccctgggtgtgaaacagctgattgttggtgtcaacaaaatggattccaccgagccaccatacagtcagaagagatacgaggaaatcgttaaggaagtcagcacctacattaagaaaattggctacaaccctgacacagtagcatttgtgccaatttctggttggaatggtgacaacatgctggagccaagtgctaatatgccttggttcaagggatggaaagtcacccgcaaagatggcagtgccagtggcaccacgctgctggaagctttggattgtatcctaccaccaactcgtccaactgacaagcctctgcgactgcccctccaggatgtctataaaattggaggcattggcactgtccctgtgggccgagtggagactggtgttctcaaacctggcatggtggttacctttgctccagtcaatgtaacaactgaagtcaagtctgttgaaatgcaccatgaagctttgagt

SEQ ID NO: 2 - DNA, 530 bp, Kagoshima Berkshire
gctgactgatcgggagaatcagtctatcttaatcaccggagaatccggggcaggaaagactgtgaacacgaagcgtgtcatccagtactttgccacaatcgccgtcactggggagaagaagaaggaggaacctactcctggcaaaatgcaggggactctggaagatcagatcatcagtgccaaccccctgctcgaggcctttggcaacgccaagaccgtgaggaacgacaactcctctcgctttggtaaattcatcaggatccacttcggtaccactgggaagctggcttctgctgacatcgaaacatatcttctagagaagtctagagtcactttccagctaaaggcagaaagaagctaccacattttttatcagatcatgtctaacaagaagccagagctcattgaaatgctcctgatcaccaccaacccatatgactacgccttcgtcagtcaaggggagatcactgtccccagcattgatgaccaagaggagctgatggccacagatagtgccattgaaatcctgg

SEQ ID NO: 3 - DNA, 589 bp, Kagoshima Berkshire
gttgttcctttaaatatgatgttgccacaagctgcattggagactcattgcagtaatatttccaatgtgccacctacaagagagatacttcaagtctttcttactgatgtacacatgaaggaagtaattcagcagttcattgatgtcctgagtgtagcagtcaagaaacgtgtcttgtgtttacctagggatgaaaacctgacagcaaatgaagttttgaaaacgtgtgataggaaagcaaatgttgcaatcctgttttctgggggcattgattccatggttattgcaacccttgctgaccgtcatattcctttagatgaaccaattgatcttcttaatgtagctttcatagctgaagaaaagaccatgccaactacctttaacagagaagggaataaacagaaaaataaatgtgaaataccttcagaagaattctctaaagatgttgctgctgctgctgctgacagtcctaataaacattcagtgtaccagatcgaatcacaggaagggcgggactaaaggaactacaagctgttagctgatgaccaagaggagctgatggccacagatagtgccattgaaatcctgg

SEQ ID NO: 4 - DNA, 469 bp, Kagoshima Berkshire
catttatgagggctacgcgctgccgcacgccatcatgcgcctggacctggcgggccgcgatctcaccgactacctgatgaagatcctcactgagcgtggctactccttctgaccacagctgagcgcgagatcgtgcgcgacatcaaggagaagctgtgctacgtggccctggacttcgagaacgagatggcgacggccgcctcctcctcctccctggaaaagagctacgagctgccagacgggcaggtcatcaccatcggcaacgagcgcttccgctgcccggagacgctcttccagccctccttcatcggtatggagtcggcgggcattcacgagaccacctacaacagcatcatgaagtgtgacatcgacatcaggaaggacctgtatgccaacaacgtcatgtcggggggcaccactgatgaccaagaggagctgatggccacagatagtgccattgaaatcctgg

SEQ ID NO: 5 - DNA, 507 bp, Kagoshima Berkshire
tatatagaaccgaatcacgtacactgggcctgaccaagcagggccaaaacaaggcaacctaggaggttataaaataggtatacgcgcgctgacacatacatactcactacccgaacgcggggacaactagggctccgccataagccatcctttcctggtcgtcgatgttgcgggctgcagttatagggctgccaaccgccatacacaccttaccagccacttattaagttacatccacgagggctctgtaccacccctaagcagtggcagtggtagccgctgcccgcttaccctgcgcagtgttggtgctagctccgtcctaagcttccccgatagccgccgctttttacacaccatcggcggactagacaccgttggttgcagcgtaagcgtctatggtagcagctgcggcgaccgccgtgtagccagcttactacatgttagtttcagcaaccaccctgccaatacccgtgttccctactccaactctgtcggtttcagccgcag

(19) United States
(12) Patent Application Publication
(10) Pub. No.: US 2005/0112602 A1
(43) Pub. Date: May 26, 2005

(54) cDNA chip for screening specific genes and analyzing their function in swine

(76) Inventors: Chul-Wook Kim, Gyeongsangnam-do (KR); Jung-Sou Yeo, Daegu (KR); Jung-Gyu Lee, Gyeongsangnam-do (KR); Young-Min Song, Gyeongsangnam-do (KR); Kwang-Keun Cho, Seoul (KR); Ki-Hwa Chung, Gyeongsangnam-do (KR); Il-Suk Kim, Gyeonggi-do (KR); Sang-Keun Jin, Gyeongsangnam-do (KR); Su-Hyun Park, Gyeongsangnam-do (KR); Ji-Won Jung, Gyeongsangnam-do (KR); Min-Jung Lee, Gyeongsangnam-do (KR); Eun-Jung Kwon, Gyeongsangnam-do (KR); Eun-Segk Cho, Gyeongsangnam-do (KR); Hwok-Rai Cho, Gyeongsangnam-do (KR); Sun-Min Shin, Gyeongsangnam-do (KR); Hee-Sun Nam, Gyeongsangnam-do (KR); Yeon-Hee Hong, Gyeongsangnam-do (KR); Sung-Kwang Hong, Gyeongsangnam-do (KR); Yang-Su Kang, Gyeongsangnam-do (KR); Young-Joo Ha, Gyeongsangnam-do (KR); Jeong-Man Rou, Gyeongsangnam-do (KR); Suk-Chun Kwack, Gyeongsangnam-do (KR); In-Ho Choi, Seoul (KR); Byeong-Woo Kim, Gyeongsangnam-do (KR)

Correspondence Address: OSTROLENK FABER GERB & SOFFEN, 1180 AVENUE OF THE AMERICAS, NEW YORK, NY, 100368403, US

(21) Appl. No.: 10/789,723

(22) Filed: Feb. 27, 2004

(30) Foreign Application Priority Data
Nov. 24, 2003 (KR) 2003-83651

(51) Int. Cl. C12Q 1/68; C12M 1/34

(52) U.S. Cl. 435/6; 435/287.2

(57) ABSTRACT

The present invention relates to a cDNA chip for screening and function analysis of swine genes and provides a cDNA chip comprising a probe to detect marker genes specifically expressed in the muscle and fat tissues of swine, in which the probe is capable of complementarily bind to the marker genes. Also, the present invention provides expression profiles of marker genes which are related to economic traits of swine by using the cDNA chip according to the present invention. Therefore, the cDNA chip according to the present invention can be used for the comparison of genetic expression according to swine breeds and tissues, genetic mutation screening, genetic polymorphism interpretation, development of new drugs for disease treatment and disease diagnosis, swine improvement.

### TECHNICAL FIELD

The present invention relates to a cDNA chip for screening and function analysis of swine genes. More particularly, the present invention relates to a technique for screening swine genes and analyzing their functions by preparing a cDNA chip comprising a probe to detect marker genes specifically expressed in the muscle and fat tissues of swine, in which the probe comprises 4434 ESTs isolated from the tissues and is capable of complementarily binding to the marker genes, and application of the technique for swine improvement.

### BACKGROUND ART

For creation of the value added in the hog raising farm, acquisition of foreign currencies, and raise of competitiveness of the domestic hog raising industry which depends on foreign countries for feed and swine supplies, it is a necessary assignment to obtain a swine breed with excellent quality. To solve this assignment, the present inventors have screened meat quality-related specific genes in swine and made a cDNA chip using the same. In the production of transformed swines using such specific genes, and branding and popularization of the new breeds, thereby creating highly added value of the hog-raising farm, the function analysis of swine genes is an indispensable step.

For the last several years, the studies of linkage map and physical maps of the pig genome have been remarkably advanced. The PiGMaP Project was initiated in Europe and now involves 18 European labs and a total of 7 other labs from the U.S., Japan and Australia. At present, nearly 1,800 markers and genes have been mapped in pigs (Archibald et al. 1994; Marklund et al. 1996; Rohrer et al. 1996). The physical genetic map in the pig currently consists of over 600 genes. Several quantitative trait loci (QTL) scans and locations of candidate genes were found on chromosome and major genes associated with traits of economic traits in the pig were identified. The genes related to growth and back fat exist on chromosomes 3, 4, 5, 6, 7, 8, 13 and 14, the genes related to meat quality exist on chromosomes 2, 3, 4, 6, 7, 12 and 15, and the genes related to reproduction traits exist on chromosomes 4, 6, 7 and 8. In addition, ESR and PRLR, candidate genes related to litter size, FUT1, a gene for disease resistant, SLA, NRAMP, and KIT, a gene for coat color, and MSHR were identified.

Concretely looking in to the main traits of the pig, the important the growth related genes were analyzed using a Wild Boar and Large White three generation family and the analysis revealed major QTL accounting for 20% of the phenotypic variance for back fat and abdominal fat on chromosome 4. A QTL for growth was found on chromosome 13 accounting for 7% to 12% of the phenotypic variation. By candidate gene analysis, PRT1 was found to be associated with back fat and birth weight and it maps in the center of chromosome 13 by Andersson et al. The pig MHC is positioned on chromosome 7. Associations between MHC haplotypes and several traits have been reported over the years. These have been confirmed, in part, using MHC class DNA probes. Recently, QTL related to growth and back fat traits was found on chromosome 7 in Chinese crosses. The QTL for back fat and birth weight are shown to be positioned near the region of TNFA and S0102. The overall results to date suggest that at least one growth and back fat QTL exists in this region. Other results have included a growth trait QTL on chromosome 6, but it seems to be associated with the effect caused by the RYR1 gene causing malignant hyperthermia or other unknown genes around RYR1. Some similar associations have been reported for chromosomes 3, 6, 8 and 14. Additionally, according to Gerbens and Tepas, it was reported that the fatty acid absorbing protein in the heart and major genetic factors are associated with average daily gain. Other candidate genes, including Leptin CCK and CCKAR have been mapped and may prove to be associated with appetite, fatness and growth traits.

Next, in the connection with the meat quality traits, it has been known that PSE pork is caused by RYR1 on chromosome 6. This has been demonstrated to be associated with several meat quality traits related to PSE in an F2 population originating from a Pietrain background. Focus has also centered on Hampshires for the RN gene which is associated with increased glycogen content and lower pH in the meat. The RN gene has now been mapped to chromosome 15 and is located between flanking markers. Andersson and colleagues have conducted one of the most complete QTL scans for meat quality using 234 markers on 191 F2 animals for mapping. QTL for several meat quality traits (pH, water holding capacity and pigmentation) were found to be on chromosomes 2 and 12. Rothschild and colleagues report that meat color and firmness scores are associated with regions on chromosomes 4 and 7. Additional associations with meat quality traits have been reported on chromosome 7 and for number of muscle fibers on chromosome 3. The activity of Malic enzyme, a lipogenic enzyme in muscle has been shown to be associated with the SLA complex on chromosome 7. Furthermore, a major QTL for androstenone level which is associated with boar taint in the region of the SLA complex was found.

Among candidate genes investigated for muscle quality is the HABP gene which may be associated with intramuscular fat. Many genes were found for myogenin.

Next, for reproduction traits, since larger resource families and time are required to obtain information thereon, which make the study difficult, results of QTL scan for these traits are limited. Wilkie et al. reported QTL for uterine length and ovulation rate, though in different chromosomal positions. Rathje et al. reported a QTL related to ovulation rate on chromosome 8, but there were some differences from the ovulation-related QTL observed by Wilkie and colleagues. In the French QTL experiment by Milan et al., a QTL for increased litter size of one piglet was found in the same location on chromosome 8 as Rathje. The large ovulation rate-related QTL on chromosome 8 is of interest as it mapped to the same region to the Booroola gene in sheep. Interestingly, Short et al. also found significant effects of this locus for litter size in commercial lines. Limited chromosome QTL analyses for reproductive QTL have been conducted on chromosomes 4, 6, 7, 13 and 15. It has been clearly demonstrated that the estrogen gene is significantly associated with litter size. Though genetic effects vary according to the breeds, the increase is 1.15 pig/litter in Meishan synthetics and 0.42 pigs/litter in Large White lines. More recent results have demonstrated that the prolactin receptor locus is significantly associated with litter size.

Finally, for disease resistance and immune response traits, to date, QTL scans for disease resistance or immune response QTL have been limited. Some immunity related QTL have been identified. Also, a QTL for cortisol level which may be related to stress and perhaps immune response, has been mapped to the end of chromosome 7. Two alpha genes FUT1 and FUT2 on porcine chromosome 6 have been identified. Vogeli and colleagues have disclosed a marker showing a polymorphism which is closely linked to ECF18R gene in Large White, Landrace, Hampshire, Duroc and Pietrain pigs and it could be a good marker for marker assisted selection of E. coli F18 adhesion resistant animals in these breeds. Recently, it has been reported that the SLA complex on chromosome 7 is associated with resistance to infections with Trichinella spiralis but not resistance to toxoplasmosis. The NRAMP1 gene, known to be associated with resistance to Salmonella challenge in mice, has been recently mapped to pig chromosome 15. Genes associated with human disorders, which have been identified in the pig, include clotting factor IX and the hypercholesterolaemia gene.

Considering the foregoing, the present inventors made efforts to find candidate genes for genetic improvement of economic traits in swine, that is, for development of swine with excellent growth performance, meat quality, disease resistance and reproductive performance.

Up to now, several technologies to analyze gene expression at the mRNA level such as northern blotting, differential display, sequential analysis of gene expression and dot blot analysis have been used to examine the genetic difference in swine. However, these methods have disadvantages which are not suitable for simultaneous analysis of a plurality of expressed products. In recent, a new technology such as cDNA microarray to overcome such disadvantages has been developed. The cDNA microarray becomes one of the strongest means to study gene expression in various living bodies. This technology is applied to simultaneous expression of numerous genes and discovery of genes in a large scale, as well as genetic polymorphism screening and mapping of genetic DNA clone. It is a highly advanced RNA expression analysis technology to quantitatively analyze RNA transcribed from already known or not-known genes. Such microarray uses a DNA chip. The gene chip is classified into cDNA (200-500 bp) chips and oligonucleotide (15-100 bp) chips according to the nucleotide to be detected. Also, according to the preparation method, it is classified into robot printing chips such as pin microarray or ink-jet and photolithography chips using the semiconductor production process. The cDNA chip peculiarly distinguishes a gene having a complementary sequence by attaching a full-length sequence of ORF (Open Reading Frames) or EST (Expression Sequence Tags) to a slide.

### DISCLOSURE OF INVENTION

Therefore, an object of the present invention is to provide a cDNA chip comprising a probe fixed thereon to detect marker genes specifically expressed in the muscle and fat tissues of swine, thereby being applied to swine applied improvement as well as screening and function analysis of swine genes, in which the probe is capable of complementarily binding to the marker genes.

It is another object of the present invention to provide expression profiles of marker genes which are related to economic traits of swine.

It is a further object of the present invention to provide means for comparison of gene expression according to breeds and tissues in swine, genetic mutation screening, genetic polymorphism interpretation, development of a new drug for treatment of diseases and disease diagnosis using the cDNA chip according to the present invention.

According to the present invention, the above-described objects are accomplished by, to prepare a probe DNA, preparing a probe DNA comprising extracting RNA from the muscle and fat tissues of swine and prepare cDNA therefrom, cloning 4434 ESTs and analyzing and screening the nucleotide sequence in the data base, amplifying the ESTs by PCR, followed isolation and purification, and immobilizing (spotting) the product with 300 yeast control genes on a slide using a DNA chip array to prepare a DNA chip, hybridizing a target DNA prepared by binding a fluorescent material to total RNA isolated from the muscle and fat tissues of swine with the prepared probe DNA, followed by scanning and analysis of image files, and examining profiles of genes specifically expressed in the muscle and fat tissues.

The present invention comprises the steps of preparation of ESTs from the muscle and fat tissues of swine and identification of sequence information; amplification of ESTs by PCR, followed by isolation and purification; preparation of a DNA chip by immobilizing the ESTs on a slide using a DNA chip array; hybridization of a fluorescent-labeled target DNA (ESTs) from total RNA isolated from the muscle and fat tissues of swine with the probe DNA, followed by scanning and image file analysis; and examination of the expression profiles of genes specifically expressed in the muscle and fat tissues of swine.

The cDNA chip for screening and function analysis of swine genes is prepared by the following steps: preparing cDNA from total RNA isolated from the muscle and fat tissues of swine; cloning 4434 ESTs thereof and analyzing and screening the obtained sequences in the database; amplifying the ESTs by PCR, followed by isolation and purification; and spotting the 4434 ESTs on a slide using a DNA chip array to prepare a DNA chip.

The cDNA chip for screening and function analysis of swine genes according to the present invention comprises a probe capable of complementarily binding to cDNA or RNA of marker genes and a substrate on which the probe is immobilized.

According to the present invention, the probe DNA immobilized on a DNA microarray of the cDNA chip for screening and function analysis of swine comprises 4434 ESTs isolated from the muscle and fat tissues of swine.

The substrate is preferably a polymer film such as silicone wafer, glass, polycarbonate, membrane, polystyrene or polyurethane. The DNA microarray according to the present invention may be prepared by immobilizing a probe on a substrate by a conventional method for preparing a DNA microarray, including photolithography, piezoelectric printing, micro pipetting, spotting and the like. In the present invention, the spotting method is used.

The marker genes to be detected from the probe DNA immobilized on the DNA microarray of the cDNA chip for screening and function analysis of swine genes according to the present invention include 1-alpha dynein heavy chain, 19 kDa-interacting protein 3-like, actin, actin alpha 1, actin gamma 2, annexin A2, annexin V, annexin II, beta-myosin heavy chain mRNA, calpain large polypeptide L2, collagen, collagen alpha 1, collagen alpha 2, collagen alpha V, discs large (Drosophila) homologue 5, fibronectin, heparan sulfate proteoglycan 2, lamin A/C, myosin, myosin heavy chain, myotubularin related protein 4, procollagen-proline, acidic secreted protein, tropomyosin, tropomyosin alpha chain, troponin C, tubulin beta chain and vimentin, which are related to the cellular structure and motility.

The marker genes to be detected from the probe DNA immobilized on the DNA microarray of the cDNA chip for screening and function analysis of swine genes according to the present invention include aldolase A, carbonate dehydratase, cytochrome C, cytochrome C oxidase subunit L cytochrome-C oxidase, fructose-1, 6-bisphosphatase, L-lactate dehydrogenase M chain, LIM domains 1 protein, NADH dehydrogenase, NADH-ubiquinone oxidoreductase chain 1, NADH4L, octanoyltransferase (COT), phosphoarginine phosphatase, phosphoglucomutase isoform 2 mRNA, protein-tyrosine kinase, pyruvate kinase, sarcolipin, tyrosine phosphatase type IVA, UDP glucose pyrophosphorylase, glycogen phosphorylase b and superoxide dismutase, which are related to the metabolism.

The marker genes to be detected from the probe DNA immobilized on the DNA microarray of the cDNA chip for screening and function analysis of swine genes according to the present invention include elongation factor 1 alpha, elongation factor 1 alpha 1, enolase 3, repetitive DNA sequence element RPE-1, reticulum protein, ribonucleoprotein polypeptide B, ribosomal protein, ribosomal protein L18a, ribosomal protein P0, transfer RNA-Trp synthetase, translation initiation factor eif1, LIM domains 1 protein and tissue inhibitor of metalloproteinase 3, which are related to the expression of genes and proteins.

The marker genes to be detected from the probe DNA immobilized on the DNA microarray of the cDNA chip for screening and function analysis of swine genes according to the present invention include complete mitochondrial DNA, mitochondrion, potassium channel and similar to creatine kinase, which are related to the signaling and communication of cells.

The marker genes to be detected from the probe DNA immobilized on the DNA microarray of the cDNA chip for screening and function analysis of swine genes according to the present invention include protease and cystein 1, which are related to the cell division.

The marker genes to be detected from the probe DNA immobilized on the DNA microarray of the cDNA chip for screening and function analysis of swine genes according to the present invention include Interleukin-2 receptor alpha chain, Kel-like protein 23 and MHC class I SLA genomic region, which are related to the immune response.

The marker genes to be detected from the probe DNA immobilized on the DNA microarray of the cDNA chip for screening and function analysis of swine genes according to the present invention include the nucleotide sequences of growth factors I, II, III, IV and V as set forth in SEQ ID NOs: 1 to 5, which are related to growth.

Also, the present invention provides a kit for screening and function analysis of swine genes comprising the cDNA chip, Cy5-dCTP or Cy3-dCTP bound cDNA from RNA of the tissue to be screened, a fluorescence scanning system and computer analysis system.

By the method for detecting the expression profiles of specific genes using the cDNA chip for screening and function analysis of swine genes according to the present invention, it is possible to evaluate meat quality of swine by analyzing marker genes expressed in a certain cell. Also, the method can be used for development of swine with improved growth performance by using the detected growth-specific genes of swine, and for disease diagnosis of swine and development of drug by identifying the profiles of genes involved in the general mechanism and the immune response to disease resistance of cells.

### BEST MODE FOR CARRYING OUT THE INVENTION

Now, the construction of the present invention will be explained through the following Examples in detail. However, the present invention is not limited thereto.

### EXAMPLE

### Example 1

### Screening of Swine Genes and Construction of cDNA Chip for Function Analysis

In order to prepare a cDNA chip for screening and function analysis of swine genes, a probe DNA was prepared by subjecting total RNA isolated form the muscle and fat tissues of Kagoshima Berkshire to PCR to obtain 4434 ESTs, cloning the ESTs, analyzing and screening their sequences in the database, amplifying the ESTs by PCR, followed by isolation and purification, and immobilizing the product on a slide using a DNA chip array to obtain a cDNA chip for screening and function analysis of swine genes.

### Preparation Example 1

### Preparation and Array of Probe DNA

Firstly, a probe DNA, which was cDNA amplified by PCR, was prepared and attached to a slide glass. Total RNA was extracted from the muscle and fat tissues of the longissimus dorsi of Kagoshima Berkshire (body weight of 30 kg and 90 kg) using a RNA extraction kit (Qiagen, Germany) according to the manual and mRNA was extracted using an oligo (dT) column. The extracted mRNA sample was subjected to RT-PCR using SP6, T3 forward primer, T7 reverse primer (Amersham Pharmacia Biotech, England) to synthesize cDNA. The total volume of each PCR reactant was 100 μl. 100 pM of a forward primer and a reverse primer were each transferred to a 96-well PCR plate (Genetics, England). Each well contained 2.5 mM dNTP, 10×PCR buffer, 25 mM MgCl₂, 0.2 μg of DNA template, 2.5 units of Taq polymerase. PCR was performed in GeneAmp PCR system 5700 (AB Applied BioSystem, Canada) under the following conditions: total 30 cycles of 30 seconds at 94° C., 45 seconds at 58° C., and 1 minute at 72° C.

The size of the amplified DNA was identified by agarose gel electrophoresis. The PCR product was precipitated with ethanol in 96-well plate, dried and stored at −20° C.

Total 4434 cDNAs (ESTs), prepared as described above, were cloned to analyze nucleotide sequences of genes which swine has and their genetic information was identified from the database at NCBI. The genes having information were isolated and purified by PCR. The genetic locus and map for the total 4434 cDNAs (ESTs) were constructed. The total 4434 cDNAs (ESTs) and 300 yeast control genes were arrayed in an area of 1.7 cm². Then, the probe DNA was spotted on a slide glass for microscope (produced by Corning), coated with CMT-GAPSTM aminosilane using Microgrid II (Biorobotics). The probe DNA was printed onto Microgrid II using a split pin. The pin apparatus was approached to the well in the microplate to inject the solution into the slide glass (1 to 2 nL). After printing of the probe DNA, the slide was dried and the spotted DNA and the slide were UV cross-linked at 90 mJ using Stratalinker™ (Stratagene, USA), washed twice with 0.2% SDS at room temperature for 2 minutes and washed once with third distilled water at room temperature for 2 minutes. After washing, the slide was dipped in a water tank at 95° C. for 2 minutes and was blocked for 15 minutes by adding a blocking solution (a mixture of 1.0 g of NaBH₄ dissolved in 300 mL of phosphate buffer (pH7.4) and 100 mL of anhydrous ethanol). Then, the slide was washed three times with 0.2% SDS at room temperature for 1 minute and once with third distilled water at room temperature for 2 minutes and dried in the air.

The marker genes which can be detected from the probe DNA prepared from the muscle and fat tissues of swine are as follows:

1) Genes for the Cellular Structure and Motility

1-alpha dynein heavy chain, 19 kDa-interacting protein 3-like, actin, actin alpha 1, actin gamma 2, annexin A2, annexin V, annexin II, beta-myosin heavy chain mRNA, calpain large polypeptide L2, collagen, collagen alpha 1, collagen alpha 2, collagen alpha V, discs large (Drosophila) homologue 5, fibronectin, heparan sulfate proteoglycan 2, lamin A/C, myosin, myosin heavy chain, myotubularin related protein 4, procollagen-proline, acidic secreted protein, tropomyosin, tropomyosin alpha chain, troponin C, tubulin beta chain and vimentin

2) Genes for the Metabolism

Aldolase A, carbonate dehydratase, cytochrome C, cytochrome C oxidase subunit I, cytochrome-C oxidase, fructose-1,6-bisphosphatase, L-lactate dehydrogenase M chain, LIM domains 1 protein, NADH dehydrogenase, NADH-ubiquinone oxidoreductase chain 1, NADH4L, octanoyltransferase (COT), phosphoarginine phosphatase, phosphoglucomutase isoform 2 mRNA, protein-tyrosine kinase, pyruvate kinase, sarcolipin, tyrosine phosphatase type IVA, UDP glucose pyrophosphorylase, glycogen phosphorylase b and superoxide dismutase

3) Genes for the Expression of Genes and Proteins

Elongation factor 1 alpha, elongation factor 1 alpha 1, enolase 3, repetitive DNA sequence element RPE-1, reticulum protein, ribonucleoprotein polypeptide B, ribosomal protein, ribosomal protein L18a, ribosomal protein P0, transfer RNA-Trp synthetase, translation initiation factor eif1, LIM domains 1 protein and tissue inhibitor of metalloproteinase 3

4) Genes for the Signaling and Communication Cells

Complete mitochondrial DNA, mitochondrion, potassium channel and similar to creatine kinase

5) Genes for the Cell Division

Protease and cystein 1

6) Genes for the Immune Response

Interleukin-2 receptor alpha chain, Kel-like protein 23 and MHC class I SLA genomic region

7) Genes for Growth

Growth factor I, II, III, IV and V as set forth in SEQ ID NO: 1 to 5

8) Others

cDNA flj13323 fis, KIAA0182 protein, KIAA1096 protein,AC015998, AR078G01iTHYEG01S, Cn26h08.x1, COI, DJ466P17.1.1(Laforin), foocen-m, HWMO12cA.1, hypothetical protein, mandarina library, MARC 1PI, MARC 2PIG, MR1-AN0039-290800-004-a01, NIH_MGC_{—}4, NIH_MGC_{—}65, NIH_MGC_{—}77, NIH_MGC_{—}77, Peripheral blood cell cDNA library, putative, reinhardtii CC-1690, small intestine cDNA library, thymosin beta-4 mRNA, unknown, unnamed protein product, chromosome 14 DNA sequence, integrin beta-1 subunit, reinhardtii CC-1690.

### Experimental Example 1

### Expression Profile Screening of Tissue Specific Genes Using the cDNA Chip According to the Present Invention

The expression profiles of genes specifically expressed in the muscle and fat tissues of swine were examined using the cDNA chip prepared in Example 1. The muscle tissue on the longissimus dorsi area was taken from the Kagoshima Berkshires having body weights of 30 kg and 90 kg. The fat tissue was taken from the Kagoshima Berkshire having a body weight of 30 kg. The muscle and fat tissues were cut into 5˜8 mm length, frozen with liquid nitrogen and stored at −70° C.

Total RNA was isolated from 0.2 to 1.0 g of the experimental group and the control group according to the manual of Trizol™ kit (Life Technologies, Inc.) to prepare the target DNA. Trizol™ was added to the tissue in an amount of 1 mL of Trizol™ per 50 to 100 mg of tissue and disrupted using a glass-Teflon or Polytron homogenizer. The disrupted granules were centrifuged at 4° C. at a speed of 12,000 g for 10 minutes and 1 mL of the supernatant was aliquoted. 200 μl of chloroform was added to each aliquot, voltexed for 15 seconds, placed on ice for 15 minutes and centrifuged at 4° C. at a speed of 12,000 g for 10 minutes. Chloroform of the same amount was again added thereto, voltexed for 15 seconds, placed on ice for 15 minutes and centrifuged at 4° C. at a speed of 12,000 g for 10 minutes. The supernatant was transferred to a new tube. 500 μl of isopropanol was added to the tube, voltexed and placed on ice for 15 minutes. The ice was cooled and centrifuged at 4° C. at a speed of 12,000 g for 5 minutes. The supernatant was removed, mixed with 1 mL of 75% cold ethanol and centrifuged at 4° C. at a speed of 12,000 g for 5 minutes. The supernatant was removed, freeze-dried on a clean bench for 30 minutes and take into 20 μl of RNase-free water or DEPC water to dissolve RNA. The total DNA concentration was set to 40 μg/17 μl for electrophoresis.

The target DNA was prepared according to the standard first-strand cDNA synthesis. Briefly, according to the method described by Schuler (1996), 40 μg of total RNA and oligo dT-18mer primer (Invitrogen Life Technologies) were mixed, heated at 65° C. for 10 minutes and cooled at 4° C. for 5 minutes. Then, 1 μl of a mixture of 25 mM dATP, dGTP and dTTP, 1 μl of 1 mM dCTP (Promega) and 2 μl of 1 mM cyanine 3-dCTP or 2 μl of 1 mM cyanine 5-dCTP, 20 units of RNase inhibitor (Invitrogen Life Technology), 100 units of M-MLV RTase, 2 μl of 10× first strand buffer were added thereto and mixed with a pipette. The reaction mixture was incubated at 38° C. for 2 hours and the non-bound nucleotide was removed by ethanol precipitation. Here, DEPC treated sterile water was used.

The slide, prepared above, was pre-hybridized with a hybridization solution (5×SSC, 0.2% SDS, 1 mg/mL herring sperm DNA) at 65° C. for 1 hour. The target DNA labeled with cyanine 3 (Cy-3) and cyanine 5 (Cy-5) was re-suspended in 20 μl of the hybridization solution and denatured at 95° C. for 2 minutes. Then, the slide was hybridized with the solution at 65° C. overnight. The hybridization was performed in a humidity chamber covered with a cover glass (Grace Bio-Lab).

After hybridization, the slide was washed 4 times with 2×SSC, 0.1% SDS at room temperature for 5 minutes while vigorously stirred in a dancing shaker. Then the slide was washed twice with 0.2×SSC for 5 minutes and with 0.1×SSC for 5 minutes at room temperature.

The slid was scanned on ScanArray 5000 (GSI Lumonics Version 3.1) with a pixel size of 50 μm. The target DNA labeled by cyanine 3-dCTP was scanned at 565 nm and the target DNA labeled by cyanine 5-dCTP was scanned at 670 nm. Two fluorescence intensities were standardized by linear scanning of the cyanine 3-dCTP- and cyanine 5-dCTP-labeled spots. The slide was again scanned on Scanarray 4000XL with a pixel size of 10 μm. The resulting TIFF image files were analyzed on Quantarray software version 2.1 and the background was automatically subtracted. The intensity of each spot was converted into Microsoft Excel on Quantarray.

The entire expression pattern of ESM (early stage muscle) genes expressed in the early stage in the muscle and fat tissues of swine were compared with those of ASM (adult stage muscle) gene expressed in the adult stage and ESF (early stage fat) gene expressed in the early stage. The “ESM-specific” and “ASM-specific” genes are shown in Table 1 and the “ESF-specific” genes are shown in Table 2. 20 genes showed a 5 times higher expression level in ASM, as compared to ESM. Also, 18 genes showed a 5 to 10 times higher expression level in ESF, as compared to ESM, and a 5 to 10 times higher expression level in ESM, as compared to ASM.

Also, the 5 following growth specific genes specifically expressed in the muscle and fat tissues of swine were found.

| | | |
|---|---|---|
| 1. GE (growth factor) I gene: | | |
| gagaccagca aatactatgt gaccatcatt gatgccccag gacacagaga cttcatcaaa | 60 | SEQ ID NO 1 |
| aacatgatta caggcacatc ccaggctgac tgtgctgtcc tgattgttgc tgctggtgtt | 120 | |
| ggtgaatttg aagctggtat ctccaagaac gggcagaccc gcgagcatgc tcttctggct | 180 | |
| tacaccctgg gtgtgaaaca gctgattgtt ggtgtcaaca aaatggattc caccgagcca | 240 | |
| ccatacagtc agaagagata cgaggaaatc gttaaggaag tcagcaccta cattaagaaa | 300 | |
| attggctaca accctgacac agtagcattt gtgccaattt ctggttggaa tggtgacaac | 360 | |
| atgctggagc caagtgctaa tatgccttgg ttcaagggat ggaaagtcac ccgcaaagat | 420 | |
| ggcagtgcca gtggcaccac gctgctggaa gctttggatt gtatcctacc accaactcgt | 480 | |
| ccaactgaca agcctctgcg actgcccctc caggatgtct ataaaattgg aggcattggc | 540 | |
| actgtccctg tgggccgagt ggagactggt gttctcaaac ctggcatggt ggttaccttt | 600 | |
| gctccagtca atgtaacaac tgaagtcaag tctgttgaaa tgcaccatga agctttgagt | | |
| 2. GE (growth factor) II gene: | | |
| gctgactgat cgggagaatc agtctatctt aatcaccgga gaatccgggg caggaaagac | 60 | SEQ ID NO 2 |
| tgtgaacacg aagcgtgtca tccagtactt tgccacaatc gccgtcactg gggagaagaa | 120 | |
| gaaggaggaa cctactcctg gcaaaatgca ggggactctg gaagatcaga tcatcagtgc | 180 | |
| caaccccctg ctcgaggcct ttggcaacgc caagaccgtg aggaacgaca actcctctcg | 240 | |
| ctttggtaaa ttcatcagga tccacttcgg taccactggg aagctggctt ctgctgacat | 300 | |
| cgaaacatat cttctagaga agtctagagt cactttccag ctaaaggcag aaagaagcta | 360 | |
| ccacattttt tatcagatca tgtctaacaa gaagccagag ctcattgaaa tgctcctgat | 420 | |
| caccaccaac ccatatgact acgccttcgt cagtcaaggg gagatcactg tccccagcat | 480 | |
| tgatgaccaa gaggagctga tggccacaga tagtgccatt gaaatcctgg | | |
| 3. GF (growth factor) III gene: | | |
| gttgttcctt taaatatgat gttgccacaa gctgcattgg agactcattg cagtaatatt | 60 | SEQ ID NO 3 |
| tccaatgtgc cacctacaag agagatactt caagtctttc ttactgatgt acacatgaag | 120 | |
| gaagtaattc agcagttcat tgatgtcctg agtgtagcag tcaagaaacg tgtcttgtgt | 180 | |
| ttacctaggg atgaaaacct gacagcaaat gaagttttga aaacgtgtga taggaaagca | 240 | |
| aatqttgcaa tcctgttttc tgggggcatt gattccatgg ttattgcaac ccttgctgac | 300 | |
| cgtcatattc ctttagatga accaattgat cttcttaatg tagctttcat agctgaagaa | 360 | |
| aagaccatgc caactacctt taacagagaa gggaataaac agaaaaataa atgtgaaata | 420 | |
| ccttcagaag aattctctaa agatgttgct gctgctgctg ctgacagtcc taataaacat | 480 | |
| tcagtgtacc agatcgaatc acaggaaggg cgggactaaa ggaactacaa gctgttagc | | |
| 4. GF (growth factor) IV gene: | | |
| catttatgag ggctacgcgc tgccgcacgc catcatgcgc ctggacctgg cgggccgcga | 60 | SEQ ID NO 4 |
| tctcaccgac tacctgatga agatcctcac tgagcgtggc tactccttct gaccacagct | 120 | |
| gagcgcgaga tcgtgcgcga catcaaggag aagctgtgct acgtggccct ggacttcgag | 180 | |
| aacgagatgg cgacggccgc ctcctcctcc tccctggaaa agagctacga gctgccagac | 240 | |
| gggcaggtca tcaccatcgg caacgagcgc ttccgctgcc cggagacgct cttccagccc | 300 | |
| tccttcatcg gtatggagtc ggcgggcatt cacgagacca cctacaacag catcatgaag | 360 | |
| tgtgacatcg acatcaggaa ggacctgtat gccaacaacg tcatgtcggg gggcaccac | | |
| 5. GF (growth factor) V gene: | | |
| tatatagaac cgaatcacgt acactgggcc tgaccaagca gggccaaaac aaggcaacct | 60 | SEQ ID NO 5 |
| aggaggttat aaaataggta tacgcgcgct gacacataca tactcactac ccgaacgcgg | 120 | |
| ggacaactag ggctccgcca taagccatcc tttcctggtc gtcgatgttg cgggctgcag | 180 | |
| ttatagggct gccaaccgcc atacacacct taccagccac ttattaagtt acatccacga | 240 | |
| gggctctgta ccacccctaa gcagtggcag tggtagccgc tgcccgctta ccctgcgcag | 300 | |
| tgttggtgct agctccgtcc taagcttccc cgatagccgc cgctttttac acaccatcgg | 360 | |
| cggactagac accgttggtt gcagcgtaag cgtctatggt agcagctgcg gcgaccgccg | 420 | |
| tgtagccagc ttactacatg ttagtttcag caaccaccct gccaataccc gtgttcccta | 480 | |
| ctccaactct gtcggtttca gccgcag | | |

TABLE 1
Expression ratio of differentially expressed genes between ESM
and ASM

| | | | |
|---|---|---|---|
| | | | Ratio of |
| ESTs | Accession | | gene expression |
| No. | No.† | Description** | ESM (30)/ASM (90) |

Cellular structure and motility

| | | | |
|---|---|---|---|
| SM2149 | CAB56598 | 1-alpha dynein heavy chain | −2.1 |
| SM781 | NP_033891 | 19 kDa-interacting protein 3- | +2.1 |
| | | like | |
| SM635 | BAB19361 | Actin | +3.4 |
| SM713 | AAA51586 | Actin | +6.3 |
| SM106 | P53506 | Actin | +8.8 |
| SM1068 | AAF20165 | Actin | +5.3 |
| SM363 | B25819 | Actin | +4.3 |
| SM768 | X52815 | Actin | +3.4 |
| SMk77 | NM_001100 | Actin, alpha 1 | +15.1 |
| SM128 | NP_033740 | Actin, gamma 2 | +6.9 |
| SM902 | BC001748 | Annexin A2 | −3.2 |
| SM846 | P81287 | Annexin V | −2.8 |
| SM653 | P04272 | Annexin II | −2.2 |
| SMk340 | U75316 | Beta-myosin heavy chain mRNA | +3.0 |
| SM1605 | AAF99682 | Calpain large polypeptide L2 | +4.7 |
| SM541 | NP_000079 | Collagen | −3.2 |
| SM715 | L47641 | Collagen | −6.8 |
| SM430 | Q9XSJ7 | Collagen alpha 1 | −6.8 |
| SM758 | CGHU1S | Collagen alpha 1 | −2.1 |
| SM62 | CGHU2V | Collagen alpha 2 | −3.2 |
| SM949 | O46392 | Collagen alpha 2 | −3.3 |
| SM410 | CAA28454 | Collagen (alpha V) | −2.3 |
| SM1651 | XM_039583 | Discs, large (Drosophila) | −2.0 |
| | | homolog 5 | |
| SM1050 | AAA30521 | Fibronectin | −2.4 |
| SM491 | NM_005529 | Heparan sulfate proteoglycan 2 | −2.2 |
| SM1573 | XM_044160 | Lamin A/C | +2.6 |
| SMk55 | NP_006462 | Myosin | +3.9 |
| SMk338 | P79293 | Myosin heavy chain | +2.0 |
| SMk168 | AB025261 | Myosin heavy chain | +9.0 |
| SM1732 | NP_004678 | Myotubularin related protein 4 | +3.8 |
| SM1691 | NP_000908 | Procollagen-proline | −2.3 |
| SM690 | NP_003109 | Secreted protein, acidic | −4.4 |
| SMk173 | X66274 | Tropomyosin | +2.6 |
| SM141 | CAA38179 | Tropomyosin | +2.7 |
| SMk51 | P18342 | Tropomyosin alpha chain | +9.6 |
| SM1043 | P06469 | Tropomyosin alpha chain | +11.5 |
| SMk19 | P02587 | Troponin C | +14.5 |
| SMk50 | Y00760 | Troponin-C | +19.6 |
| SMk57 | AAA91854 | Troponin-C | +14.6 |
| SM1535 | P02554 | Tubulin beta chain | +2.8 |
| SM1063 | P20152 | Vimentin | −5.4 |

Metabolism

| | | | |
|---|---|---|---|
| SMk56 | AAA37210 | Aldolase A | +5.5 |
| SM995 | CAA59331 | Carbonate dehydratase | +3.2 |
| SMk344 | NM_012839 | Cytochrome C | +3.4 |
| SM800 | AAG53955 | Cytochrome c oxidase subunit I | +3.0 |
| SM51 | T10974 | Cytochrome-c oxidase | +3.8 |
| SMk151 | CAA06313 | Fructose-1,6-bisphosphatase | +7.1 |
| SM2070 | P00339 | L-lactate dehydrogenase M chain | +12.7 |
| SMk120 | AJ275968 | LIM domains 1 protein | +8.6 |
| SMk147 | X59418 | NADH dehydrogenase | +2.4 |
| SM928 | O79874 | NADH-ubiquinone oxidoreductase | +5.3 |
| | | chain 1 | |
| SMk18 | AAG28185 | NADH4L | +2.1 |
| SMk81 | O19094 | Octanoyltransferase(COT) | +3.2 |
| SM295 | AB006852 | Phosphoarginine phosphatase | +2.6 |
| SMk346 | M97664 | Phosphoglucomutase isoform 2 mRNA | +5.5 |
| SM36 | TVMVRR | Protein-tyrosine kinase | +4.3 |
| SM887 | P11980 | Pyruvate kinase | +8.5 |
| SM698 | S64635 | Pyruvate kinase | +9.7 |
| SM723 | P52480 | Pyruvate kinase | +7.3 |
| SMk79 | U44751 | Pyruvate kinase | +5.2 |
| SMk135 | Z98820 | Sarcolipin | +3.0 |
| SM1033 | XM_018138 | Tyrosine phosphatase type IVA | +2.9 |
| SMk347 | X99312 | UDP glucose pyrophosphorylase | +3.0 |

Gene/protein expression

| | | | |
|---|---|---|---|
| SM75 | U09823 | Elongation factor 1 alpha | −4.3 |
| SM1989 | AAH05660 | Elongation factor 1 alpha 1 | −3.9 |
| SMk61 | NP_031959 | Enolase 3 | +3.6 |
| SM968 | Y00104 | Repetitive dna sequence element | −2.5 |
| | | RPE-1 | |
| SMk91 | AAC48501 | Reticulum protein | +4.6 |
| SM2083 | NP_003083 | Ribonucleoprotein polypeptide B | +3.1 |
| SM896 | AAH01127 | Ribosomal protein | +2.0 |
| SM1668 | AAH07512 | Ribosomal protein L18a | +2.1 |
| SM1784 | 228176 | Ribosomal protein P0 | +6.2 |
| SM1801 | AAA30799 | Transfer RNA-Trp synthetase | +6.0 |
| SM997 | 51077272 | Translation initiation factor | +3.5 |
| | | eif1 | |

Cell signaling/communication

| | | | |
|---|---|---|---|
| SM464 | AJ002189 | Complete mitochondrial DNA | +3.9 |
| SM732 | AF304203 | Mitochondrion | +5.9 |
| SMk11 | XM_006515 | Potassium channel | −2.4 |
| SMk187 | BC007462 | Similar to creatine kinase | +3.5 |

Cell division

| | | | |
|---|---|---|---|
| SM1067 | XP_007399 | Protease, cysteine, 1 | +3.1 |

Immune response

| | | | |
|---|---|---|---|
| SM154 | AF036005 | Interleukin-2 receptor alpha | −2.5 |
| | | chain | |
| SMk1 | AAAG52886 | Kel-like protein | +6.4 |
| SM401 | AJ251829 | MHC class I SLA genomic region | −3.0 |

EST

| | | | |
|---|---|---|---|
| SM824 | AK023385 | cDNA FLJ13323 fis | +2.5 |
| SM1776 | XM_050494 | KIAA0182 protein | +3.6 |
| SM1556 | XP_043678 | KIAA1096 protein | +4.9 |

Unknown

| | | | |
|---|---|---|---|
| SM1785 | AC015998 | AC015998 | +2.1 |
| SM2152 | BI327422 | AR078G01iTHYEG01S | −4.0 |
| SM1469 | BG938561 | Cn26h08.x1 | −2.2 |
| SM908 | AAG28205 | COI | +2.8 |
| SM851 | AAG28192 | COI | +3.6 |
| SM1738 | CAA19420 | DJ466P17.1.1(Laforin) | +4.8 |
| SM1007 | AAD31021 | Foocen-m | +3.8 |
| SM1920 | BE421626 | HWM012cA.1 | +3.3 |
| SM1972 | XP_039195 | Hypothetical protein | +3.2 |
| SM1536 | T08758 | Hypothetical protein | +4.7 |
| SMk137 | XP_002275 | Hypothetical protein | +20.0 |
| SM1724 | XP_016035 | Hypothetical protein | −2.6 |
| SM1539 | AT001097 | Mandarina library | −2.3 |
| SM1474 | BG384994 | MARC 1PI | +2.6 |
| SM1853 | BF198401 | MARC 2PIG | +3.6 |
| SM1941 | BE925069 | MR1-AN0039-290800-004-a01 | +4.4 |
| SM379 | AW328623 | NIH_MGC_4 | +2.3 |
| SM1911 | BE872239 | NIH_MGC_65 | −2.4 |
| SM1676 | BG548727 | NIH_MGC_77 | +5.1 |
| SM1914 | BG534187 | NIH_MGC_77 | −2.3 |
| SM1650 | BI337009 | Peripheral Blood Cell cDNA | +9.3 |
| | | library | |
| SM1064 | BAB28119 | Putative | +3.4 |
| SM618 | BAB28422 | Putative | +2.1 |
| SM1774 | BAB30715 | Putative | +3.2 |
| SM1690 | BF864360 | Reinhardtii CC-1690 | +2.2 |
| SM1898 | F23148 | Small intestine cDNA library | −2.3 |
| SM96 | M17733 | Thymosin beta-4 mRNA | −4.2 |
| SM1922 | AAH03026 | Unknown | +4.0 |
| SM210 | BAA91923 | Unnamed protein product | −3.1 |

No match

| | | |
|---|---|---|
| SM107 | No match | −2.4 |
| SM278 | No match | −2.2 |
| SM384 | No match | −2.3 |
| SMk37 | No match | +7.7 |
| SM717 | No match | −3.0 |
| SM1598 | No match | +4.5 |
| SMk6 | No match | +3.8 |
| SMk68 | No match | +5.0 |
| SM1100 | No match | −2.6 |
| SMk70 | No match | +3.9 |
| SMk80 | No match | +17.7 |
| SMk112 | No match | +3.5 |
| SM1639 | No match | −4.0 |
| SMk148 | No match | +3.8 |
| SM1665 | No match | +3.8 |
| SM1665 | No match | +13.0 |
| SMk95 | No match | +2.7 |
| SMk133 | No match | +2.4 |
| SMk152 | No match | +6.4 |
| SM1897 | No match | +3.4 |
| SMk138 | No match | +10.3 |
| SM1902 | No match | +2.1 |
| SMk342 | No match | +6.7 |
| SMk181 | No match | +11.0 |
| SM904 | No match | −3.4 |
| SMk262 | No match | +3.9 |
| SM9 | No match | +2.4 |
| SM1964 | No match | +2.6 |
| SMk335 | No match | −3.9 |

†agreed Accession no.
**Information agreed to the database
No match: No information agreed to the database; novel EST
ESM: early stage muscle (body weight 30 kg),
ASM: adult stage muscle (body weight 90 kg),
SM: swine muscle

TABLE 2
Expression ratio of differentially expressed genes between ESM
and ESF

| | | | |
|---|---|---|---|
| | | | Ratio of |
| ESTs | Accession | | gene expression |
| No. | No†. | Description** | ESF (30)/ESM (30) |

Cellular structure and motility

| | | | |
|---|---|---|---|
| SM2149 | CAB56598 | 1-alpha dynein heavy chain | −2.1 |
| SM781 | NP_033891 | 19 kDa-interacting protein 3- | +2.2 |
| | | like | |
| SM1068 | AAF20165 | Actin | +4.5 |
| SM635 | BAB19361 | Actin | +2.6 |
| SM106 | P53506 | Actin | +4.9 |
| SM768 | X52815 | Actin | +2.4 |
| SM363 | B25819 | Actin | +3.7 |
| SM713 | AAA51586 | Actin | +5.6 |
| SMk77 | NM_001100 | Actin, alpha 1 | +4.5 |
| SM128 | NP_033740 | Actin, gamma 2 | +3.9 |
| SM1091 | JC5971 | Alpha-b crystallin | +2.1 |
| SM902 | BC001748 | Annexin A2 | −4.2 |
| SM846 | P81287 | Annexin V | −3.5 |
| SM653 | P04272 | Annexin II | −2.3 |
| SMk340 | U75316 | Beta-myosin heavy chain mRNA | +2.2 |
| SM1807 | AAF99682 | Calpain large polypeptide L2 | +2.7 |
| SM541 | NP_000079 | Collagen | −4.9 |
| SM715 | L47641 | Collagen | −5.2 |
| SM1023 | Q9XSJ7 | Collagen alpha 1 | −4.6 |
| SM758 | CGHU1S | Collagen alpha 1 | −4.3 |
| SM62 | CGHU2V | Collagen alpha 2 | −4.4 |
| SM949 | O46392 | Collagen alpha 2 | −3.2 |
| SM410 | CAA28454 | Collagen(alpha V) | −2.3 |
| SM1121 | NM_000393 | Collagen, type V, alpha 2 | −2.8 |
| SM53 | NP_000384 | Collagen, type V, alpha 2 | −2.5 |
| SM1651 | XM_039583 | Discs, large(Drosophila) | −8.6 |
| | | homolog 5 | |
| SM1050 | AAA30521 | Fibronectin | −3.1 |
| SM381 | FNHU | Fibronectin precursor | −2.6 |
| SM122 | P07589 | Fibronectin(FN) | −2.5 |
| SM1573 | XM_044160 | Lamin A/C | +2.1 |
| SMk55 | NP_006462 | Myosin | +3.6 |
| SMk168 | AB025261 | Myosin heavy chain | +5.0 |
| SM1732 | NP_004678 | Myotubularin related protein 4 | +4.7 |
| SM690 | NP_003109 | Secreted protein, acidic | −5.2 |
| SM1043 | P06469 | Tropomyosin alpha chain | +8.6 |
| SMk173 | X66274 | Tropomysin | +2.2 |
| SMk19 | P02587 | Troponin C | +6.9 |
| SMk57 | AAA91854 | Troponin-C | +7.1 |
| SMk50 | Y00760 | Troponin-C | +9.0 |
| SM1535 | P02554 | Tubulin beta chain | +3.3 |
| SM1063 | P20152 | Vimentin | −5.1 |
| SM730 | CAA69019 | Vimentin | −3.2 |

Metabolism

| | | | |
|---|---|---|---|
| SMk344 | NM_012839 | Cytochrome C | +2.4 |
| SM800 | AAG53955 | Cytochrome c oxidase subunit I | +2.9 |
| SMk151 | CAA06313 | Fructose-1,6-bisphosphatase | +4.2 |
| SMk254 | 231300 | Glycogen Phosphorylase b | +2.6 |
| SM2070 | P00339 | L-lactate dehydrogenase M chain | +10.6 |
| SM928 | O79874 | NADH-ubiquinone oxidoreductase | +3.2 |
| | | chain 1 | |
| SMk81 | O19094 | Octanoyltransferase(COT) | +3.9 |
| SM295 | AB006852 | Phosphoarginine phosphatase | +2.3 |
| SMk346 | M97664 | Phosphoglucomutase isoform 2 mRNA | +3.3 |
| SM36 | TVMVRR | Protein-tyrosine kinase | +2.6 |
| SM723 | P52480 | Pyruvate kinase | +7.5 |
| SM698 | S64635 | Pyruvate kinase | +6.6 |
| SM887 | P11980 | Pyruvate kinase | +6.3 |
| SM1594 | AAA62278 | Superoxide dismutase | −3.2 |
| SM1033 | XM_018138 | Tyrosine phosphatase type IVA | +2.2 |

Gene/protein expression

| | | | |
|---|---|---|---|
| SM75 | U09823 | Elongation factor 1 alpha | −3.7 |
| SM1989 | AAH05660 | Elongation factor 1 alpha 1 | −3.8 |
| SMk120 | AJ275968 | LIM domains 1 protein | +9.9 |
| SMk91 | AAC48501 | Reticulum protein | +2.1 |
| SM2083 | NP_003083 | Ribonucleoprotein polypeptide B | +3.2 |
| SM21 | NP_000994 | Ribosomal | +2.2 |
| SM1784 | 228176 | Ribosomal protein P0 | +5.5 |
| SM1820 | BC014277 | Tissue inhibitor of | −2.6 |
| | | metalloproteinase 3 | |
| SM1801 | AAA30799 | Transfer RNA-Trp synthetase | +5.7 |
| SM997 | 51077272 | Translation initiation factor | +2.3 |
| | | eif1 | |

Cell signaling/communication

| | | | |
|---|---|---|---|
| SM464 | AJ002189 | Complete mitochondrial DNA | +2.7 |

Immune response

| | | | |
|---|---|---|---|
| SMk1 | AAG52886 | Kel-like protein 23 | +4.6 |

EST

| | | | |
|---|---|---|---|
| SM1776 | XM_050494 | KIAA0182 | +3.2 |
| SM1556 | XP_043678 | KIAA1096 protein | +4.5 |

Unknown

| | | | |
|---|---|---|---|
| SM2152 | BI327422 | AR078G01iTHYEG01S | −5.5 |
| SMk3 | AL13277 | Chromosome 14 DNA sequence | +2.3 |
| SM908 | AAG28205 | COI | +2.2 |
| SM1738 | CAA19420 | DJ466P17.1.1(Laforin) | +3.5 |
| SM1007 | AAD31021 | Foocen-m | +3.0 |
| SM1724 | XP_016035 | Hypothetical protein | −2.6 |
| SMk137 | XP_002275 | Hypothetical protein | +10.0 |
| SM1972 | XP_039195 | Hypothetical protein | +2.8 |
| SM787 | AF192528 | Integrin beta-1 subunit | +2.0 |
| SM1474 | BG384994 | MARC 1PI | +2.8 |
| SM1676 | BG548727 | NIH_MGC_77 | +2.3 |
| SM1650 | BI337009 | Peripheral Blood Cell cDNA | +7.3 |
| | | library | |
| SM1774 | BAB30715 | Putative | +5.1 |
| SM1064 | BAB28119 | Putative | +3.0 |
| SM1690 | BF864360 | Reinhardtii CC-1690 | +2.5 |
| SM96 | M17733 | Thymosin beta-4 mRNA | −3.9 |
| SM1922 | AAH03026 | Unknown | +4.7 |

No match

| | | |
|---|---|---|
| SMk58 | No match | +2.9 |
| SM717 | No match | −4.4 |
| SMk6 | No match | +2.4 |
| SMk68 | No match | +3.2 |
| SMk80 | No match | +4.3 |
| SMk112 | No match | +2.1 |
| SM1639 | No match | −2.8 |
| SMk148 | No match | +2.9 |
| SM1665 | No match | +9.8 |
| SMk95 | No match | +2.1 |
| SMk152 | No match | +6.4 |
| SM1897 | No match | +2.6 |
| SMk138 | No match | +3.1 |
| SM796 | No match | −2.2 |
| SMk342 | No match | +3.9 |
| SMk181 | No match | +4.4 |
| SM904 | No match | −2.7 |
| SMk262 | No match | +2.7 |
| SM9 | No match | +2.9 |
| SM1964 | No match | +2.6 |
| SMk335 | No match | +3.8 |

†agreed Accession no.
**Information agreed to the database
No match: No information agreed to the database; novel EST
ESM: early stage muscle (body weight 30 kg),
ESF: early stage fat (body weight 30 kg),
SM: swine muscle

From the above results, the present inventors shown the expression profiles of genes specifically expressed in the muscle and fat tissues of swine using the cDNA for screening and function analysis of swine genes according to the present invention and present the usability thereof in the improvement and evaluation of meat quality. Also, we identified the nucleotide sequences of growth-related factors and present the applicability thereof in the development of swine with excellent growth performance. In addition, by using the cDNA chip according to the present invention, it is expectedly possible to screen and compare expression profiles of genes according to swine breeds and tissues and to perform genetic mutation screening, genetic polymorphism interpretation, development of new drugs for disease treatment and disease diagnosis.

### Example 2

### Construction of a Kit for Screening and Function Analysis of Swine Genes

A kit for screening and function analysis of swine genes comprising the cDNA chip fabricated in Example 1, Cy5-dCTP or Cy3-dCTP bound cDNA from RNA of the tissue to be screened, a fluorescence scanning system and a computer analysis system was fabricated.

Industrial Applicability

As explained through the Examples, the present invention relates to a cDNA chip for screening and function analysis of swine genes and provides a cDNA chip comprising a probe to detect marker genes specifically expressed in the muscle and fat tissues of swine, in which the probe is capable of complementarily bind to the marker genes. Also, the present invention provides expression profiles of marker genes which are related to economic traits of swine by using the cDNA chip according to the present invention. Therefore, the cDNA chip according to the present invention can be used for the comparison of genetic expression according to swine breeds and tissues, genetic mutation screening, genetic polymorphism interpretation, development of new drugs for disease treatment and disease diagnosis, swine improvement and thus, is very useful invention for the genetic engineering industry.

1. A cDNA chip for screening and function analysis of swine genes comprising a probe capable of detecting marker genes specifically expressed in the muscle and fat tissues of swine and a substrate on which the probe is immobilized.

2. The cDNA chip according to claim 1, wherein the probe includes 4434 ESTs (Expression Sequence Tags) derived from the muscle and fat tissues of swine for the marker genes.

3. The cDNA chip according to claim 1, wherein the marker genes to be detected from the probe which are related to the cellular structure and motility include 1-alpha dynein heavy chain, 19 kDa-interacting protein 3-like, actin, actin alpha 1, actin gamma 2, annexin A2, annexin V, annexin II, beta-myosin heavy chain mRNA, calpain large polypeptide L2, collagen, collagen alpha 1, collagen alpha 2, collagen alpha V, Discs, large (Drosophila) homolog 5, fibronectin, heparan sulfate proteoglycan 2, lamin A/C, myosin, myosin heavy chain, myotubularin related protein 4, procollagen-proline, acidic secreted protein, tropomyosin, tropomyosin alpha chain, troponin C, tubulin beta chain and vimentin.

4. The cDNA chip according to claim 1, wherein the marker genes to be detected from the probe which are related to the metabolism include aldolase A, carbonate dehydratase, cytochrome C, cytochrome C oxidase subunit I, cytochrome-C oxidase, fructose-1,6-bisphosphatase, L-lactate dehydrogenase M chain, LIM domains 1 protein, NADH dehydrogenase, NADH-ubiquinone oxidoreductase chain 1, NADH4L, octanoyltransferase (COT), phosphoarginine phosphatase, phosphoglucomutase isoform 2 mRNA, protein-tyrosine kinase, pyruvate kinase, sarcolipin, tyrosine phosphatase type IVA, UDP glucose pyrophosphorylase, glycogen phosphorylase b and superoxide dismutase.

5. The cDNA chip according to claim 1, wherein the marker genes to be detected from the probe which are related to the expression of genes and proteins include elongation factor 1 alpha, elongation factor 1 alpha 1, enolase 3, repetitive DNA sequence element RPE-1, reticulum protein, ribonucleoprotein polypeptide B, ribosomal protein, ribosomal protein L18a, ribosomal protein P0, transfer RNA-Trp synthetase, translation initiation factor eif1, LIM domains 1 protein and tissue inhibitor of metalloproteinase 3.

6. The cDNA chip according to claim 1, wherein the marker genes to be detected from the probe which are related to the signaling and communication of cells include complete mitochondrial DNA, mitochondrion, potassium channel and similar to creatine kinase.

7. The cDNA chip according to claim 1, wherein the marker genes to be detected from the probe which are related to the cell division include protease and cystein 1.

8. The cDNA chip according to claim 1, wherein the marker genes to be detected from the probe which are related to the immune response include Interleukin-2 receptor alpha chain, Kel-like protein 23 and MHC class I SLA genomic region.

9. The cDNA chip according to claim 1, wherein the marker genes to be detected from the probe which are related to growth include the nucleotide sequences of growth factors I, II, III, IV and V as set forth in SEQ ID NOs: 1 to 5.

10. A kit for screening and function analysis of swine genes comprising the cDNA chip as defined in claim 1.
